(19) United States
(12) Patent Application Publication
(10) Pub. No.: US 2008/0208352 A1
(43) Pub. Date: Aug. 28, 2008

(54) Stent Having Controlled Porosity for Improved Ductility

(75) Inventors: Michael Krivoruchko, Forestville, CA (US); Jeffrey Allen, Santa Rosa, CA (US); Matthew J. Birdsall, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/679,426

(22) Filed: Feb. 27, 2007

(51) Int. Cl. A61F 2/86

(52) U.S. Cl. 623/23.7

(57) ABSTRACT

An endoluminal prosthesis for placement in a body lumen of a metallic material having controlled porosity for improved ductility. The metallic material may be formed into a stent structure or a wire or sheet, which may then be formed into the stent structure. The porous network of the stent includes pores that range from nanometer scale to micron scale. The controlled porosity accommodates volume changes as well as provides a barrier to crack propagation to allow alloy steels and amorphous metal materials, which would otherwise be considered too brittle for the demands of intraventional use, to be utilized in a stent.

### FIELD OF THE INVENTION

The invention relates generally to endoluminal prostheses for placement in a body lumen, and more particularly to stents made of a metallic material having a controlled porosity that provides ductility for improved performance.

### BACKGROUND OF THE INVENTION

A wide range of medical treatments exist that utilize “endoluminal prostheses.” As used herein, endoluminal prostheses is intended to cover medical devices that are adapted for temporary or permanent implantation within a body lumen, including both naturally occurring and artificially made lumens, such as without limitation: arteries, whether located within the coronary, mesentery, peripheral, or cerebral vasculature; veins; gastrointestinal tract; biliary tract; urethra; trachea; hepatic shunts; and fallopian tubes.

Accordingly, a wide assortment of endoluminal prostheses have been developed, each providing a uniquely beneficial structure to modify the mechanics of the targeted lumen wall. For example, stent prostheses are known for implantation within body lumens to provide artificial radial support to the wall tissue, which forms the various lumens within the body, and often more specifically, for implantation within the blood vessels of the body.

To provide radial support to a vessel, such as one that has been widened by a percutaneous transluminal coronary angioplasty, commonly referred to as “angioplasty,” “PTA” or “PTCA”, a stent is implanted in conjunction with the procedure. Effectively, the stent must overcome the natural tendency of the vessel walls of some patients to close back down. As such, the stent acts as a scaffolding to resist the vessels tendency to close back down. Under this procedure, the stent may be collapsed to an insertion diameter and inserted into a body lumen at a site remote from the diseased vessel. The stent may then be delivered to the desired treatment site within the affected lumen and deployed, by self-expansion or radial expansion, to its desired diameter for treatment.

In certain instances due to the stretching of the vessel wall that occurs during a PTCA procedure, the stretching and widening of the vessel to reopen the lumen and the subsequent making of the vessel patent for facilitating revascularization of the heart tissue can result in vessel injury at the treatment site. The resulting trauma to the vessel wall contributes to the extent and occurrence of restenosis of the vessel. A problem associated with stent expansion at the treatment site is that the stent may need to be over expanded in order to compensate for high metallurgical recoil, which occurs in many stents made of high strength materials, such as, stainless steel, MP35N, ELGILOY, nitinol, L605, magnesium, niobium, and tantalum. This over expansion can contribute to the trauma that occurs to the vessel wall.

Accordingly, a vascular stent must possess a unique set of properties so that it can travel through small and tortuous body lumens to the treatment site, as well as be expanded to no more than its working diameter to provide consummate lumen expansion and radial support subsequent to implantation. Ideally, the stent should be formed from a material that exhibits a high tensile strength but that provides flexibility to the stent for navigating the tortuous vascular anatomy. Further, a radially-expandable stent must undergo significant plastic deformation when being expanded into its deployed state, which requires a stent material to have good elongation or ductility. Finally, an ideal stent material should have a high degree of radiopacity, good corrosion resistance and biocompatibility to vascular tissue, blood and other bodily fluids. However, these requirements are often competing and/or contradictory, such that a sacrifice or trade-off between one or more properties is customarily required in choosing a stent material.

Stents are typically constructed from metal alloys that include any of stainless steel, nickel-titanium (NiTi or nitinol), cobalt-chromium (MP35N), platinum, and other suitable metals. Customarily such commercially available materials are designed for one or two properties, e.g., strength and endurance, at the sacrifice of others, e.g., formability and/or processability. However, there is an ever present need in the art for developing a vascular stent that is made from a material and by a method that imparts many of the desired properties to the stent with minimal trade-offs.

### BRIEF SUMMARY OF THE INVENTION

Embodiments of the present invention include an endoluminal prosthesis for placement in a body lumen of a metallic material having controlled porosity for improved ductility. The metallic material may be formed into a stent structure or a wire or sheet, which may then be formed into the stent structure. The porous network of the stent includes pores that range from nanometer scale to micron scale. The controlled porosity accommodates volume changes as well as provides a barrier to crack propagation to allow alloy steels and amorphous metal materials, which would otherwise be considered too brittle for the demands of intraventional use, to be utilized in a stent.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 a perspective view of an exemplary stent in accordance with an embodiment of the present invention.

FIG. 1A is a magnified view of a cross-sectional portion of a stent strut taken along line A-A of FIG. 1.

### DETAILED DESCRIPTION OF THE INVENTION

The following detailed description is merely exemplary in nature and is not intended to limit the invention or the application and uses of the invention. Although the description of embodiments of the invention may be in the context of treatment of blood vessels, the invention may also be used in any other body passageways where it is deemed useful. Furthermore, there is no intention to be bound by any expressed or implied theory presented in the preceding technical field, background, brief summary or the following detailed description.

Many high strength materials, such as alloy steels and amorphous metals, exhibit poor ductility that limits there ability to be used in vascular stent applications. In accordance with embodiments of the present invention, in order to improve ductility, these materials may be formed into a wire, sheet or tube having a 3-D porous network with pores that range from nanometer scale to micron scale. The pores allow volume changes as well as provide a barrier to crack propagation. The wire, sheet or tube having controlled porosity, i.e., a select quantity and distribution of a pore size or sizes, may then be formed into a vascular device according to embodiments of the present invention that may be made from alloy steels and amorphous metal materials, which would otherwise be considered too brittle for the demands of interventional use. The vascular device thus formed is able to undergo a high degree of strain during loading and deployment without fracture or high recoil. In an embodiment, a pore size in the range of 10 nm to 25 μm is preferred for imparting improved ductility to elemental or an alloy, for example, MP35N or other nickel-cobalt-chromium-molybdenum alloy, a cobalt-based alloy, such as, HAYNES® alloy 25 (L605), and 316L stainless steel alloy. An increase in ductility is especially desirable for brittle alloys or elemental materials, such as those of or including iridium, magnesium and zinc, for example. In another embodiment, a unitary stent structure of a metallic material having controlled porosity may be formed in a molding process as discussed further below.

Powder metallurgical technologies are well suited for the manufacturing of metallic stents with controlled porosity, wherein porosity is measured as the total volume of pores of the sintered metal divided by the total volume of the metal. A powder metallurgy process begins with the selection of an elemental or alloy metal powder and, in some cases, one or more binders or additives that are suitable for forming pores. A particle size or sizes of the metal powder may be selected to correlate with a desired porosity in the finished product. If a pore forming additive or spacer material is used, the particle size of the additive may be selected to define the controlled porosity in the finished product. In such an embodiment, the metal powder and pore forming additive are mixed together to form a particulate mixture.

The metal powder or particulate mixture is then subjected to a forming process for creating the wire, sheet or tube from which the stent structure will ultimately be formed or for creating the stent structure. Various powder metallurgy forming processes include isostatic pressing (hot or cold), die compacting, injection molding, extruding, spraying, plasma texturing, rolling, slip or tape casting, vapor deposition and pressureless sintering. Wet powder spraying (WPS), tape casting and a space holder process are suitable processes for controlling the porosity of a structure formed by powder metallurgy.

Wet powder spraying may be used to achieve a graded porous structure, such as a graded porous metallic tube or sheet that may than be formed into a stent. Tape casting permits the manufacturing of self-supporting 2-D sheets of a metallic material with thicknesses of between 12 μm to greater than 3 mm that may than be formed into a stent. In the wet powder spraying and tape casting processes, the resulting porosity of the stent may be adjusted by selection of an appropriate particle size distribution in the metal powder or particulate mixture. As well, sintering conditions may be adjusted to provide for a given porosity. Depending on the initial powder, a controlled porosity in the finished stent may be achieved in the range of 20-50% volume with a pore size of from 0.1 to 200 μm. In a preferred embodiment, a pore size in a range of 10 nm to 10 μm is desirable to provide improved ductility to a stent made of an amorphous material.

A space holder process with a suitable spacer or pore forming material may be used to provide a controlled porosity in the finished stent of up to 80% volume. Porosities between 40 and 80% volume as well as pore sizes up to 2 mm can be adjusted by the amount of the spacer material and/or the level of fractionating of its particle size during mixing. The production of semi-finished wires, sheets or tubes may be performed by multiaxial or cold isostatic pressing. Shaping is followed by removal of the spacer material and subsequent sintering at temperatures between 900 and 1300° C. depending on the properties of the metal powder. The metallic wires, sheets or tubes having controlled porosity may then be formed into a stent by methods discussed below.

Metal injection molding (“MIM”), which comprises compounding, molding, de-binding, and sintering, is another powder metallurgy process that may be used to create the stent structure, and/or the wire, sheet or tube from which the stent structure will ultimately be formed. In compounding, metal powders are combined with an appropriate pore forming material, which may be a polymer or other synthetic binder, typically in a batch mixer. The mixture is then granulated, i.e., further mixed, typically in an extruder, to form the mixture into the granules that will be fed into a molding machine. Then, the compounded powders are molded into a green part by one of, for example, injection molding, compression molding, and transfer molding. Optionally, to achieve less porosity, the binder or pore forming material may be removed from the molded green part before sintering by solvents and/or heat processes. Removing the binder before continuing sintering typically will enhance the compactness, i.e., reduce/control the porosity, of the molded structure. After de-binding, the molded stent structure is heated to a temperature below the melting temperature of the metal alloys to enable a re-flow of the metal alloys, i.e., sintering, wherein pressure may be applied to further reduce/control the porosity of the stent structure. To maintain or achieve an increased porosity, de-binding may be performed after sintering and/or minimum or no pressure may be applied during the sintering process. Further by alternating compounding conditions, e.g., powder/binder ratio, sizes of the powder, and sintering conditions, e.g., temperature, duration, and pressures, various configurations and sizes of pores may be produced in the finished stent structure.

In accordance with various embodiments of the present invention, metal powders of high strength materials, such as alloy steels and amorphous metals, may be selected from a group of biocompatible metals, for example, iridium, magnesium, iron and zinc. The metals or alloys may be selected to optimize, for example: manufacturability, e.g., injection molding, laser welding, heat treatment and other secondary operations, compatibility with the deployment methods, e.g., ease of transformation between the unexpanded and expanded forms, flexibility for maneuvering through the tortuous pathway, capability of withstanding radial compression force from the lumen, and versatility in design.

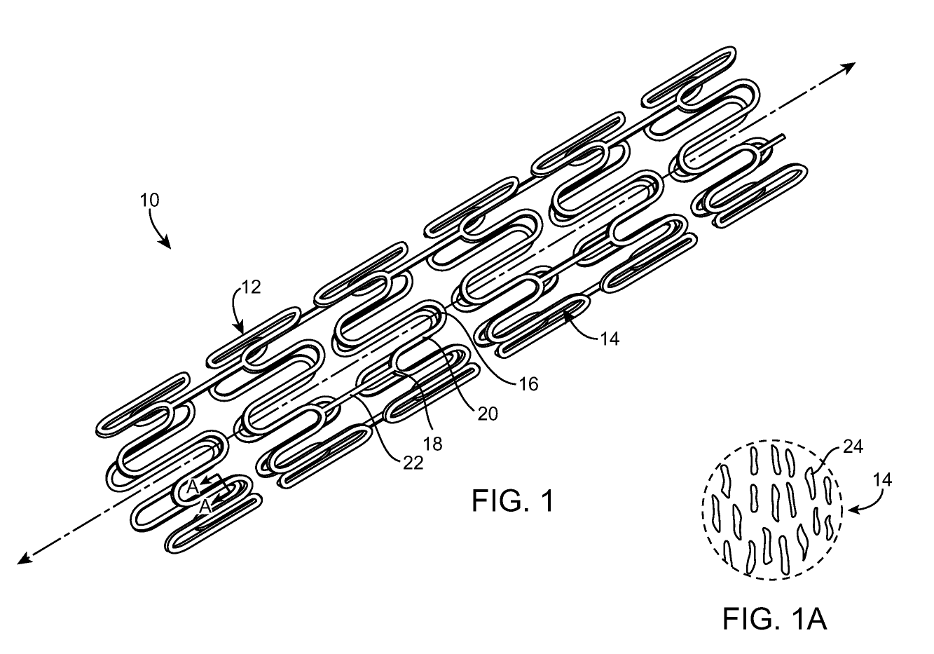

FIGS. 1 and 1A illustrate an endoluminal prosthesis in accordance with an embodiment of the present invention. Stent 100 is a patterned tubular device of a metallic material having controlled porosity that includes a plurality of radially expandable cylindrical rings 12. FIG. 1A is a magnified view of a cross-sectional portion of a stent strut 14 taken along line A-A of FIG. 1 that illustrates pores 24 evenly distributed through the metallic material thereof. In another embodiment, a random orientation of pores 24 exists within the metallic material of stent 100. Pores 24 may be present throughout the metallic material that makes up the structure of stent 100 and thus are present from an inner surface to an outer surface of stent 100, in other words throughout an entire thickness of the stent. In another embodiment, a discrete layer having pores 24 extending a quarter to half way through a thickness of the stent structure may be sufficient to provide a barrier to crack propagation in the material of the stent thereby improving the ductility of the stent structure and avoiding failure.

Cylindrical rings 12 are formed from struts 14 in a generally sinusoidal pattern including peaks 16, valleys 18, and generally straight segments 20 connecting peaks 16 and valleys 18. Connecting links 22 connect adjacent cylindrical rings 12 together. In FIG. 1, connecting links 22 are shown as generally straight links connecting peak 16 of one ring 12 to valley 18 of an adjacent ring 12. However, connecting links 22 may connect a peak 16 of one ring 12 to a peak 16 of an adjacent ring, or a valley 18 to a valley 18, or a straight segment 20 to a straight segment 20. Further, connecting links 22 may be curved. Connecting links 22 may also be excluded, with a peak 16 of one ring 12 being directly attached to a valley 18 of an adjacent ring 12, such as by welding, soldering, or the manner in which stent 100 is formed, such as by etching the pattern from a flat sheet or a tube made from a metallic material having controlled porosity.

It will be appreciated by those of ordinary skill in the art that stent 100 of FIG. 1 is merely an exemplary stent and that stents of various forms may be used in accordance with various embodiments of the present invention. In an embodiment, a stent may be formed from a thin-walled, small diameter metallic tube made from a metallic material having controlled porosity that is cut to produce the desired stent pattern, using methods such as laser cutting or chemical etching. The cut stent may then be de-scaled, polished, cleaned and rinsed. In another embodiment, a stent may be formed from one or more wires of a metallic material having controlled porosity shaped into a zig-zag, peak and valley or wire mesh arrangement. Some examples of methods of forming stents and structures for stents are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,776,161 to Globerman, U.S. Pat. No. 5,935,162 to Dang, U.S. Pat. No. 6,090,127 to Globerman, U.S. Pat. No. 6,113,627 to Jang, U.S. Pat. No. 6,663,661 to Boneau and U.S. Pat. No. 6,730,116 to Wolinsky et al., each of which is incorporated by reference herein in its entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. An endoluminal prostheses for placement in a body lumen comprising:
a stent of a metallic material having controlled porosity with pores in a range of 10 nm to 10 μm, wherein the metallic material is an amorphous metal alloy and the controlled porosity of the amorphous metal alloy provides ductility to the stent.

2. The endoluminal prosthesis of claim 1, wherein the amorphous metal alloy includes at least one of iridium, magnesium and zinc.

3. The endoluminal prosthesis of claim 1, wherein the controlled porosity is throughout a thickness of the stent.

4. The endoluminal prosthesis of claim 1, wherein the controlled porosity is in an outer half of a thickness of the stent.

5. The endoluminal prosthesis of claim 1, wherein the controlled porosity is in an outer quarter of a thickness of the stent.

6. An endoluminal prostheses for placement in a body lumen comprising:
a stent of a metallic material having controlled porosity with pores in a range of 10 nm to 10 μm, wherein the metallic material is an alloy steel and the controlled porosity of the alloy steel provides ductility to the stent.

7. The endoluminal prosthesis of claim 6, wherein the alloy steel is selected from a group consisting of MP35N or other nickel-cobalt-chromium-molybdenum alloy, a cobalt-based steel alloy, an L605 alloy, and a 316L stainless steel alloy.

8. The endoluminal prosthesis of claim 6, wherein the controlled porosity is throughout a thickness of the stent.

9. The endoluminal prosthesis of claim 6, wherein the controlled porosity is in an outer half of a thickness of the stent.

10. The endoluminal prosthesis of claim 6, wherein the controlled porosity is in an outer quarter of a thickness of the stent.
